SEQUENCE LISTING (2 sequences)

SEQ ID NO: 1 - DNA, 22 bp, artificial sequence
  antisense oligonucleotide of trkA
catcaacgaagtcaccagaccg

SEQ ID NO: 2 - DNA, 22 bp, artificial sequence
  mismatch oligonucleotide of trkA
caacatcgaagtgacgagaccg

(19) United States
(12) Patent Application Publication
(10) Pub. No.: US 2011/0020362 A1
(43) Pub. Date: Jan. 27, 2011

(54) Adrenergic stimulation mediates hypersensitivity by inducing nerve growth factor expression

(76) Inventor: Sushil K. Sarna, Houston, TX (US)

Correspondence Address: Benjamin Aaron Adler;ADLER & ASSOCIATES, 8011 Candle Lane, Houston, TX, 77071, US

(21) Appl. No.: 12/804,332

(22) Filed: Jul. 20, 2010

Related U.S. Application Data
(60) Provisional application No. 61/271,466, filed on Jul. 21, 2009.

(51) Int. Cl. A61K 39/395; A61K 31/36; A61K 31/553; A61K 31/7088; A61K 38/12; A61K 31/417; A61K 31/138; A61K 31/404; A61P 1/00

(52) U.S. Cl. 424/158.1; 514/450; 514/211.08; 514/44 A; 514/21.1; 514/401; 514/652; 514/415

(57) ABSTRACT

The current invention provides mechanisms by which chronic stress induced hypersensitivity exacerbates the symptoms of irritable bowel syndrome. The invention includes methods of treating irritable bowel syndrome using agents which inhibit key molecules involved in the sensitization pathway.

### CROSS REFERENCE TO RELATED APPLICATION

This non-provisional application claims the benefit of priority under 35 U.S.C. §119(e) of provisional U.S. Ser. No. 61/271,466, filed Jul. 21, 2009, now abandoned, the entirety of which is hereby incorporated by reference.

### FEDERAL FUNDING LEGEND

This invention was created in part using funds from the federal government under grant DK 032346 and DK 072414 from the National Institute of Disease and Digestive and Kidney Diseases. Consequently, the federal government has certain rights in this invention.

### BACKGROUND OF THE INVENTION

1. Field of the Invention

The present invention generally relates to the field of irritable bowel syndrome (IBS) physiology and therapeutics. Specifically, the present invention relates to mechanisms of how chronic stress exacerbates symptoms of irritable bowel syndrome.

2. Description of the Related Art

Abdominal discomfort/cramping and altered bowel habits are the defining symptoms of irritable bowel syndrome. The etiologies of both symptoms are not fully understood. However, clinical studies show that chronic stress can exacerbate or precipitate both symptoms of irritable bowel syndrome (1-2). This suggests that chronic stress may impair the cellular functions of some of the same cells that cause motility dysfunction or visceral hypersensitivity in irritable bowel syndrome patients in the first place. However, the mechanisms by which chronic stress causes cellular dysfunction in these cells may differ from those that cause the underlying dysfunction in irritable bowel syndrome.

Animal studies have supported this hypothesis. A recent study found that alterations in transcription of genes encoding key cell-signaling proteins of excitation-contraction coupling in colonic circular smooth muscle cells underlie motility dysfunction of faster colonic transit and increase in defecation rate in a model of post-infective irritable bowel syndrome (3). Another study found that chronic stress enhances transcription of some of the same genes encoding key cell-signaling proteins that accelerate colonic transit and defecation rate, but by different mechanisms (4).

Clinical studies show that primary spinal afferents mediate visceral hypersensitivity to colorectal distension (CRD) in irritable bowel syndrome patients (5-6). On the other hand, all psychological stress responses begin in the central nervous system (CNS). The release of corticotrophin releasing hormone (CRH) from the paraventricular nucleus of the hypothalamus is an early and essential step in initiation of all psychological stress responses (7). The central release of CRH and other mediators, such as angiotensin, vasopressin, stimulate the neuroendocrine system comprised of autonomic neurons and the hypothalamus-pituitary-adrenal (HPA-axis), which modulates the adaptive and maladaptive responses of peripheral organs in a stress- and cell-type specific manner. Both acute and chronic stressors induce visceral hypersensitivity to colorectal distension in rats by releasing CRH in the hypothalamus (8-9). However, it is unclear which pathways or stress mediators of the neuroendocrine system transmit the central signal to colon-specific primary afferent neurons in the dorsal root ganglia (DRG) to modulate their sensitivity to colorectal distension.

There is still, therefore, a recognized need in the art for improved understanding of dorsal root ganglia sensitization. Specifically, the prior art is deficient in understanding the specific mechanisms by which chronic stress may exacerbate or release symptoms of abdominal pain/cramping in irritable bowel syndrome patients. The present invention fulfills this long-standing need in the art as well as provide therapeutic targets which help alleviate the symptoms of irritable bowel syndrome.

### SUMMARY OF THE INVENTION

The present invention is directed to a method of treating an individual with irritable bowel syndrome, comprising reducing hypersensitivity of dorsal root ganglia neurons in the individual.

The present invention is also directed to a method of treating an individual with irritable bowel syndrome, comprising inhibiting nerve growth factor in the individual.

The present invention is further directed to a method of treating an individual with irritable bowel syndrome, comprising administering inhibitor of norepinephrine to the individual.

The present invention is further directed to a method of treating an individual with irritable bowel syndrome, comprising: reducing hypersensitivity of dorsal root ganglia neurons in the individual; inhibiting nerve growth factor in the individual; administering an inhibitor of norepinephrine to the individual; or a combination thereof.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIGS. 1A-1B show heterotypic chronic stress significantly increased viscero-motor response to graded colorectal distension, n=14, *p<0.05 compared to pre-stress baseline responses 8-hours following the last stressor (FIG. 1A). No significant increase was observed at 24-hours and 7-days after heterotypic chronic stress. Sham stressed control rats showed no change in their viscero-motor response, n=10. (FIG. 1B).

FIGS. 2A-2E show the effects of heterotypic chronic stress and colonic resiniferatoxin treatment on electrophysiological properties of DiI labeled thoracolumbar (TL) colon dorsal root ganglia neurons. Changes in electrophysiological properties of colon specific dorsal root ganglia neurons from control (Ctr, n=31), chronic stress (heterotypic chronic stress, n=22) rats, chronic stress rats pretreated with resiniferatoxin (RTX, n=23) and control rats treated with resiniferatoxin (n=20). (FIG. 2A) Current clamp traces showing action potentials induced by current injection at rheobase. (FIG. 2B) Graph showing changes in Rheobase. (FIG. 2C) Graph showing changes in resting membrane potential. (FIG. 2D) Current clamp traces showing the number of action potentials at 2× rheobase. (FIG. 2E) Number of action potentials (AP) induced by current injection at 2× rheobase. *p<0.05, control vs heterotypic chronic stress, #p<0.05 heterotypic chronic stress vs heterotypic chronic stress+RTX, +p<0.05, heterotypic chronic stress vs control+RTX.*

FIGS. 3A-3E show (FIG. 3A) heterotypic chronic stress significantly increased nerve growth factorin muscularis externa/serosa and mucosa/submucosa, which was blocked by inhibition of α1/α2 and β1/β2-adrenergic receptors prior to each daily stress session. n=8, *p<0.01. (FIG. 3B) Immunohistochemical staining for nerve growth factor (brown) in distal colon cross-sections from control and stressed rats. Sections were counterstained with hematoxilyn. (FIG. 3C) nerve growth factorantagonism by systemic administration of neutralizing antibody significantly reduced heterotypic chronic stress-induced increase in viscero-motor response heterotypic chronic stress, n=4, heterotypic chronic stress+nerve growth factorAb, n=5, *p<0.05 baseline vs post-heterotypic chronic stress; post-heterotypic chronic stress vs post-HeCS+nerve growth factorAb. +p<0.05. (FIG. 3D) Intrathecal administration of trkA antagonist k252A (n=5) or anti-sense ODN (n=3) suppressed heterotypic chronic stress-induced increase in viscero-motor response. *p<0.05 baseline vs post-heterotypic chronic stress, +p<0.05, post-heterotypic chronic stress+MM vs post-heterotypic chronic stress+nerve growth factorAb. (FIG. 3E) Western blot showing the effects of intrathecal treatment with either trkA AS or MM ODN on trkA expression in thoracolumbar (TL), Lumbar-Sacral (LS) or lower thoracic DRG. n=4, *p<0.05.

FIGS. 4A-4C show intrathecal treatment with trkA antagonist k252a blocked the heterotypic chronic stress-induced increase in excitability of TL colon dorsal root ganglia neurons (Ctr=control, n=13; heterotypic chronic stress, n=22; heterotypic chronic stress pre=treated with RTX, n=23. (FIG. 4A) Rheobase. (FIG. 4B) Resting membrane potential. (FIG. 4C) Number of action potentials (AP) at 2× rheobase *p<0.05, control vs heterotypic chronic stress, #p<0.05 heterotypic chronic stress vs heterotypic chronic stress+k252a.

FIGS. 5A-5B show electrophysiological properties of TL colon dorsal root ganglia neurons that were incubated for 24 hours with either high nerve growth factor (250 ng/mL or low nerve growth factor (2.5 ng/mL) in vitro. Neurons incubated with high nerve growth factor showed a significant decline in resting membrane potential (FIG. 5A) and rheobase (FIG. 5B), *p<0.05.

FIG. 6 shows daily systemic administration of α1/α2- and β1/β2-adrenergic receptor antagonists phentolamine and propranolol respectively prior to each daily stress session blocked the heterotypic chronic stress-induced increase in the viscero-motor response in response to graded colorectal distension (n=3).

FIGS. 7A-7B shows in vitro incubation of muscularis externa/serosa (FIG. 7A) and mucusa/submucosa (FIG. 7B) for 24-hours with norepinephrine concentration-dependently increased the expression of nerve growth factor. n=6, *p<0.05.

FIGS. 8A-8E show that (FIG. 8A) heterotypic chronic stress did not increase MPO in the muscularis externa/serosa or mucosa/submucosa. (FIG. 8B) and (FIG. 8C) heterotypic chronic stress did not increase the spontaneous or ionomycininduced release of tryptase from the muscularis externa/serosa or the mucosa/submucosa. Instead, it decreased the spontaneous release of tryptase in the muscularis externa/serosa. n=4, *p<0.05. (FIG. 8D) and (FIG. 8E). CRH1/CRH2 receptor antagonist astressin (AST) had no effect on the norepinephrine-induced expression of nerve growth factorin the muscularis externa/serosa, but it blocked this increase in mucosa/submucosa. n=4, *p<0.05.

### DETAILED DESCRIPTION OF THE INVENTION

As used herein, the term “a” or “an”, when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, method steps, and/or methods of the invention. It is contemplated that any device, compound, composition, or method described herein can be implemented with respect to any other device, compound, composition, or method described herein.

As used herein, the term “or” in the claims refers to “and/or” unless explicitly indicated to refer to alternatives only or the alternatives are mutually exclusive, although the disclosure supports a definition that refers to only alternatives and “and/or”.

In one embodiment of the present invention, there is provided a method of treating an individual with irritable bowel syndrome by reducing hypersensitivity of dorsal root ganglia neurons in the individual. The reduction may be achieved by administering resiniferatoxin. For example, resiniferatoxin may be administered luminally in the distal colon. A representative dosage may be around 200 ug/kg.

In another embodiment of the present invention, there is provided a method of treating an individual with irritable bowel syndrome, by inhibiting nerve growth factor in the individual. The inhibition may be achieved by administering an nerve growth factor antibody or inhibitor of trkA receptors or blocking α1/α2- and β1/β2/β3-adrenergic receptors. Inhibitor of trkA receptors may be k252A, astressin or trkA anti-sense oligonucleotide. The sequence of the anti-sense oligonucleotide is CATCAACGAAGTCACCAGACCG (SEQ ID NO: 1). The inhibitors may be administered intrathecally.

In another embodiment of the present invention, there is provided a method of treating an individual with irritable bowel syndrome, by administering inhibitor of norepinephrine to the individual. The inhibitor may be phentolamine, propranolol, cyanopindolol or a combination thereof. The inhibitor may be injected intraperitoneally. A person having ordinary skill in this art would be readily able to determine an appropriate dosage of the inhibitor such as, for example, 2 mg/kg.

In yet another embodiment of the present invention, there is provided a method of treating an individual with irritable bowel syndrome, comprising: reducing hypersensitivity of dorsal root ganglia neurons in the individual; inhibiting nerve growth factor in the individual; administering an inhibitor of norepinephrine to the individual; or a combination thereof. Methods of reducing hypersensitivity of dorsal root ganglia neurons, inhibiting nerve growth factor or administering an inhibitor of norepinephrine are described in detail herein.

In a recent study (4), it was discovered that nine-day heterotypic chronic stress (HeCS) significantly increases the plasma concentration of norepinephrine. However, the actual mechanism of norepinephrine release was not determined. In the present study, it is considered that norepinephrine released by the central component of stress response acts as a messenger to induce expression of neurotrophin, nerve growth factor (NGF), in the distal colon, which sensitizes the colon-specific neurons in the dorsal root ganglia (DRG) to induce visceral hypersensitivity to colorectal distension. Chronic stress was chosen because clinical findings indicate that chronic stress, rather than short-term acute stress (1-2) exacerbates the symptoms of irritable bowel syndrome. In addition, variable stressors are less likely to show adaptation compared to repeated applications of the same stressor.

### Example 1

### Animals

Male Wistar rats between 6 and 10 weeks of age were used. Rats were housed at a constant temperature with a standard light/dark cycle. The Institutional Animal Care and Use Committee at University of Texas Medical Branch (UTMB) approved all procedures performed on animals.

### Example 2

### Heterotypic Chronic Stress (HeCS) Protocol

Rats were subjected to nine consecutive days of a heterotypic stress protocol comprised of three randomly arranged stressors, 60-minutes of water avoidance stress (WAS), 45-minutes of cold restraint stress (CRS) at 4° C. or 20-minutes of forced swimming stress (FSS), as described (4). Stress sessions were performed in the following order: CRS, FSS, WAS, CRS, WAS, FSS, WAS, FRS, CRS.

### Example 3

### Measurement of Viscero-Motor Response (VMR) to Graded CRD

Two electrodes were implanted under general 2% isofluorane anesthesia in the external oblique abdominal muscle and exteriorized in the subscapular region. Rats were allowed one week to recover from surgery. Under 2% isofluoruthane anesthesia, a 5 cm long balloon constructed from a surgical glove finger and attached to tygon tubing was inserted 7 cm into the descending colon and rectum. Rats were placed in small Lucite cubicles (20×8×8 cm) and allowed to adapt for 30 minutes. colorectal distension was performed by rapidly inflating the balloon to constant pressures: 20, 30, 40, 50, 60 & 80 mm Hg, for 20 seconds followed by 2-minute rest. Electromygraphic (EMG) activity from the external oblique muscle was recorded on Biopac equipment (Biopac Systmes, Inc., Santa Barbara, Calif.). The EMG signal was amplified, filtered at 300 Hz and digitized. The area under the curve (AUC) for the EMG signal during each 20 seconds of distention was calculated using Acknowledge software (Biopac Systmes, Inc). The net value for each distension period was calculated by subtracting the baseline value derived from the average AUC for 20 seconds before and 20 seconds after the distention period.

### Example 4

### Tissue Isolation

The distal colon, about 6 cm long was opened along the mesentery and dissected free of mucosa/submucosa and submucosal plexus. Tissues were either snap frozen in liquid nitrogen or washed in Hanks balanced salt solution and incubated in DME+10% fetal calf serum+penicillin/streptomycin containing norepinephrine for 24-hours.

### Example 5

### Nerve Growth Factor Expression

Tissue was homogenized in PBS+protease inhibitor cocktail purchased from Sigma and centrifuged at 13,000 g for 10 minutes. Nerve growth factor protein was measured by ELISA from R&D Systems (Minneapolis, Minn.).

### Example 6

### Electrophysiology

The lipid soluble fluorescent dye 1,1′dioleyl-3,3,3′,3′-tetramethylindocarbocyanine methanesulfonate (DiI-I), 25 mg in 0.5 mL methanol, was injected in 2 μL volumes at eight to ten sites in distal colon wall starting at the pelvic girdle and moving toward the cecum. TL dorsal root ganglia neurons were isolated from dorsal root ganglia T13, L1 and L2 were dissected out and put in an ice-cold, oxygenated dissecting solution, containing (in mM): 130 NaCI, 5 KCI, 2 KH2PO4, 1.5 CaCl2, 6 MgSO4, glucose, and 10 HEPES, pH 7.2 (osmolarity, 305 mOsm), as described (10). Connective tissue free ganglia were transferred to a 10 ml dissecting solution containing collagenase D (1.8 mg/mL, Roche) and trypsin (1.0 mg/mL; Sigma, St. Louis, Mo.), and incubated for 1.5 hours at 34.5° C. DRGs are then taken from the enzyme solution, washed, and put in 2 mL of the dissecting solution containing DNase (0.5 mg/mL; Sigma). Cells were subsequently dissociated by trituration with fire-polished glass pipettes and placed on acid-cleaned glass cover slips. Dorsal root ganglia neurons containing the retrograde label were identified as bright red neurons using fluorescent microscopy with a rhodamine filter (excitation 546, barrier filter at 580) and Hoffman contrast optics. For current clamp experiment, cells were superfused (1.5 mL/min) at room temperature with control external solution containing (in mM): 130 NaCI, 5 KCI, 2 KH2PO4, 2.5 CaCl2, 1 MgCl2, 10 HEPES, and 10 glucose, pH adjusted to 7.4 with NaOH (295-300 mOsm). Recording pipettes pulled from borosilicate glass tubing had resistance of 1-4 MΩ. For perforated patch recording, pipette tip was initially filled with amphotericin-free pipette solution, containing (in mM): 100 KmeSO3, 40 KCI, and 10 HEPES, pH 7.25 adjusted with KOH (osmolarity, 290 mOsm). The pipette was then backfilled with same pipette solution containing amphotericin B (300 μg/ml). Whole cell currents and voltage were recorded with Dagan 3911 patch clamp amplifier; and data were acquired and analyzed by pCLAMP 9.2 (Axon Instruments). The currents were filtered at 2-5 kHz and sampled at 50 or 100 μsec per point.

### Example 7

### Intrathecal Catheter/Osmotic Pumps

A catheter (ReCathco) was inserted through the occipital membrane onto the top of the spinal cord. The end of the catheter rested at about L1. After measurement of baseline response to colorectal distension, the catheter was primed with the appropriate solution and connected to subcutaneously implanted osmotic pump (Durect Corporation, Cupertino, Calif.) filled with appropriate solution. The pump delivered a constant infusion of 0.93 μL/hour for 10.6 days. K252a, a non-specific antagonist of tyrosine kinase receptors (trk) including the high affinity trkA receptors (11), was dissolved in DMSO and administered in sterile saline containing 10% DMSO at a concentration of 0.067 mg/mL. Each rat received an initial bolus infusion of 0.67 μg followed by 1.5 μg/day from the osmotic pump. Previously validated anti-sense and missense oligonucleotides were used (12), TrkA antisense: CATCAACGAAGTCACCAGACCG (SEQ ID NO: 1) and mismatch: CAACATCGAAGTGACGAGACCG (SEQ ID NO: 2). Oligos were HPLC purified and purchased from Invitrogen. Rats received an initial bolus dose of 10 nmole in 10 uL. The catheter was attached to the pump, which delivered oligo at a rate of 0.5 nmole/hour. Stress protocol started 24-hours after attachment of the pump. Nerve growth factor neutralizing antibody (16 μg/kg i.p. in 0.5) was purchased from R&D systems (Minneapolis, Minn.) and reconstituted in phosphate buffered saline (PBS). Control rats received PBS containing an equal concentration of goat non-immune serum.

### Example 8

### Pharmacological Agents

Propanalol, phentolamine, cyanopindolol, RU486 and norepinephrine were purchased from Sigma. A solution of propanalol and phentoalamine was prepared fresh each day in water. RU486 was prepared as a suspension in olive oil and injected subcutaneously.

### Example 9

### Myeloperoxidase and Tryptase Measurements

MPO protein in colon tissues was measured in extracts prepared in PBS+protease inhibitors by ELISA (Hycult Biotechnology, Uden, The Netherlands). To measure tryptase, portions of distal colon muscularis externa or mucosa/submucosa was incubated for 25 minutes in oxygenated Krebs at 37° C. Incubation medium was centrifuged and tryptase activity measured using tosyl-gly-pro-lys-pNA as a substrate (Millipore, Billerica, Mass.).

### Example 10

### Statistics

Data are expressed as mean±SE. One-way ANOVA followed by Fisher posthoc analysis and t-test were used for comparison of means. The effect of HeCS and treatments were analyzed using two-way repeated measures ANOVA.

### Example 11

### Chronic Stress-Induced Visceral Hypersensitivity is Associated with Increase in Excitability of Colon-Specific TL dorsal root ganglia Neurons

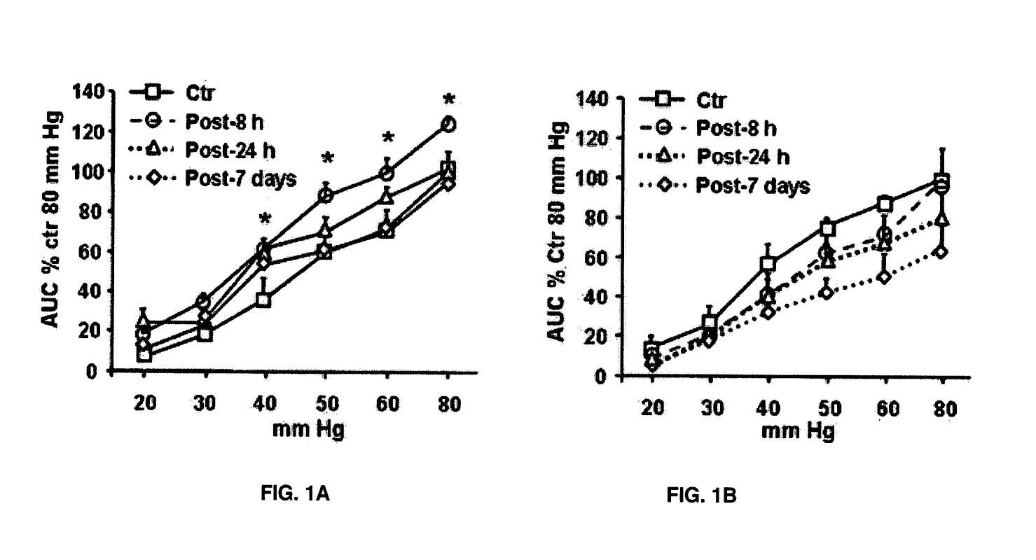

A 9-day heterotypic chronic stress significantly increased viscero-motor response to graded colorectal distension at pressures of 40, 50, 60 and 80 mm Hg, compared to pre-stressed baseline viscero-motor response (FIG. 1A). The increase in viscero-motor response persists for at least 8-hours after the last stressor, but it returns to basal levels 24-hours post-HeCS.

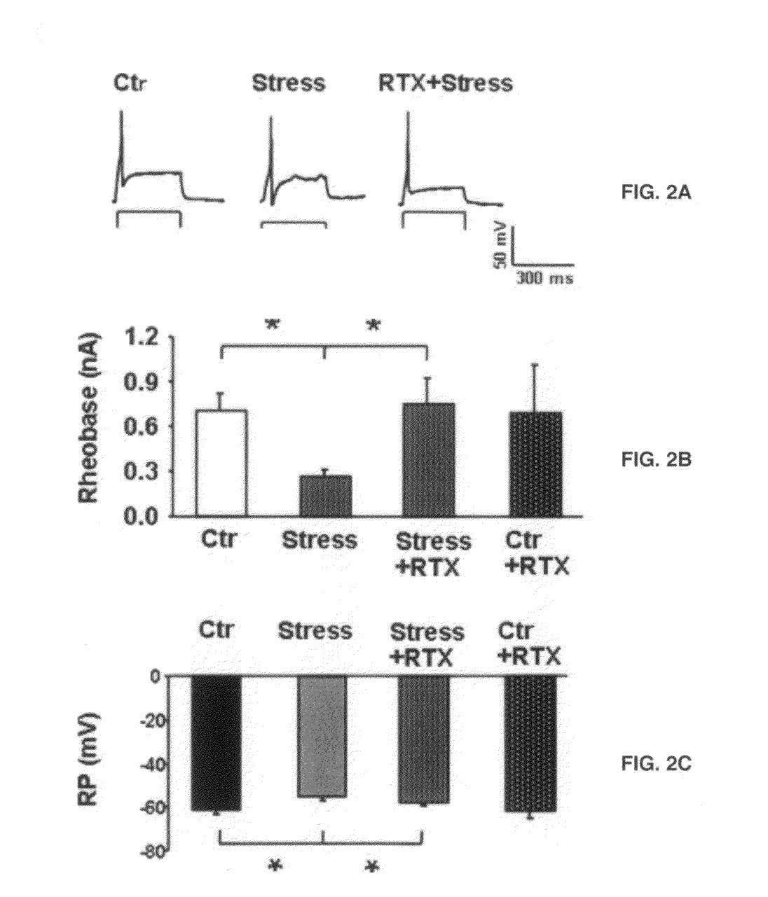

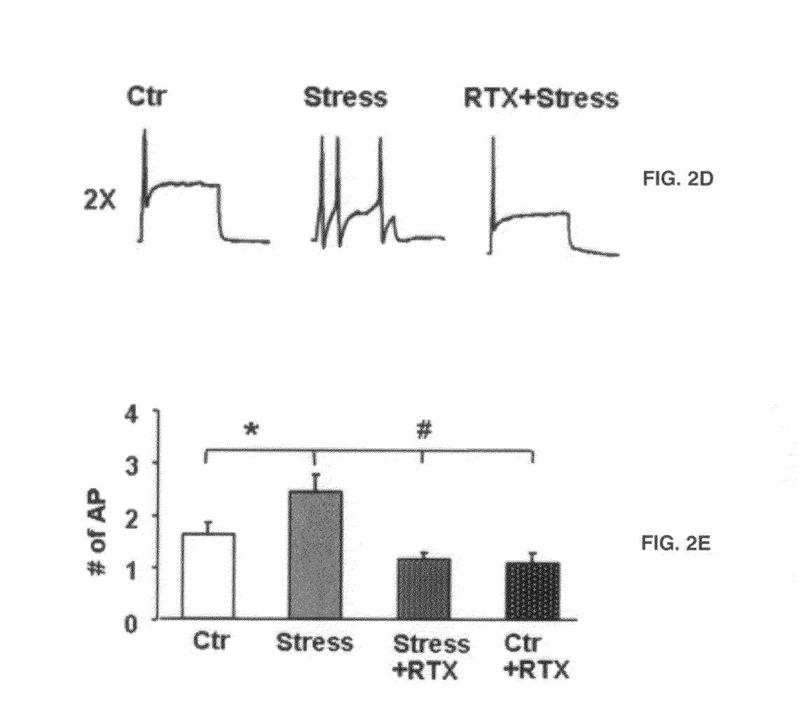

In contrast, 9-day sham stress in age matched control rats has no significant effect on viscero-motor response (FIG. 1B). Acutely dissociated TL colon-specific afferent neurons, identified by the presence of retrograde label DiI injected into the colon wall, showed a significant decrease in Rheobase (FIGS. 2A-2B), depolarization of resting membrane potential (FIG. 2C), and a significant increase in the number of action potentials evoked at 2× rheobase (FIGS. 2D-2E), compared to neurons from the sham-stressed control rats. HeCS did not alter the other electrophysiological characteristics, such as cell diameter, capacitance, input resistance and action potential amplitude, duration, threshold and overshoot, of dorsal root ganglia neurons. The role of afferent nerve endings in the above remodeling of dorsal root ganglia was investigated by desensitizing them by intraluminal infusion of 200 μg/kg resiniferatoxin (13) in the distal colon, one-day prior to beginning of the heterotypic chronic stress protocol. Treatment of sham stressed rats with resiniferatoxin had no significant effect on rheobase and resting membrane potential of colon-specific dorsal root ganglia neurons (FIGS. 2B-2C). However, in rats subjected to heterotypic chronic stress, resiniferatoxin-treatment significantly blocked the reduction in rheobase and increase in the number of action potentials at 2× (FIGS. 2A-2B, 2D-2E).

### Example 12

### HeCS Induces Nerve Growth FactorExpression in the Distal Colon

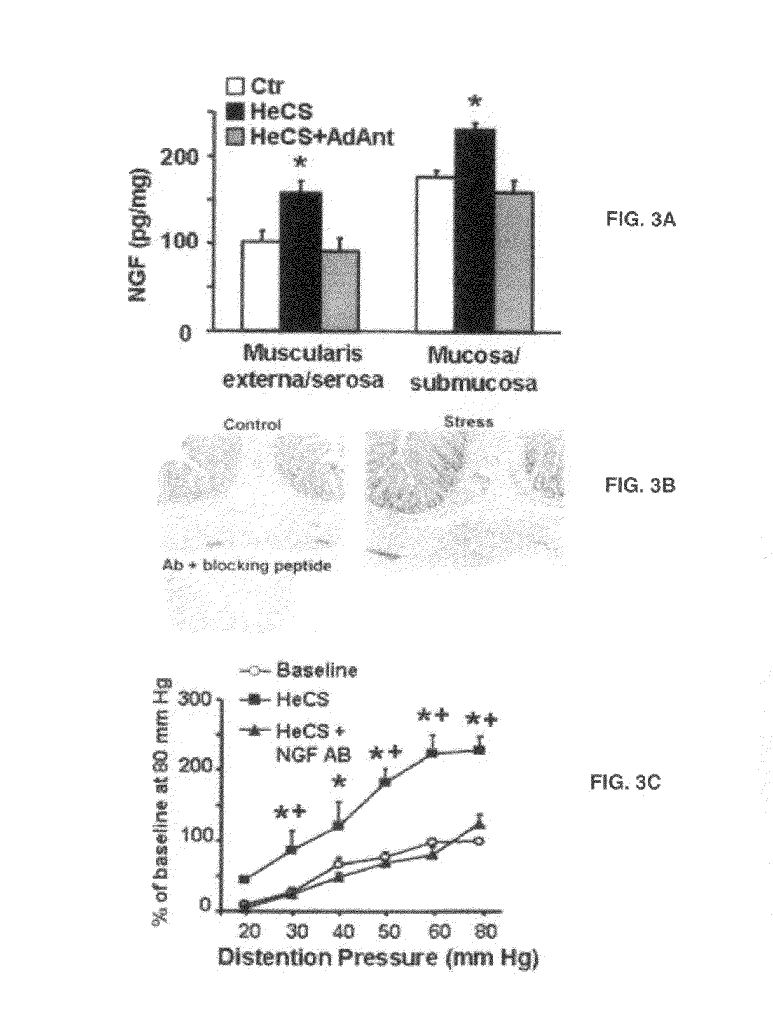

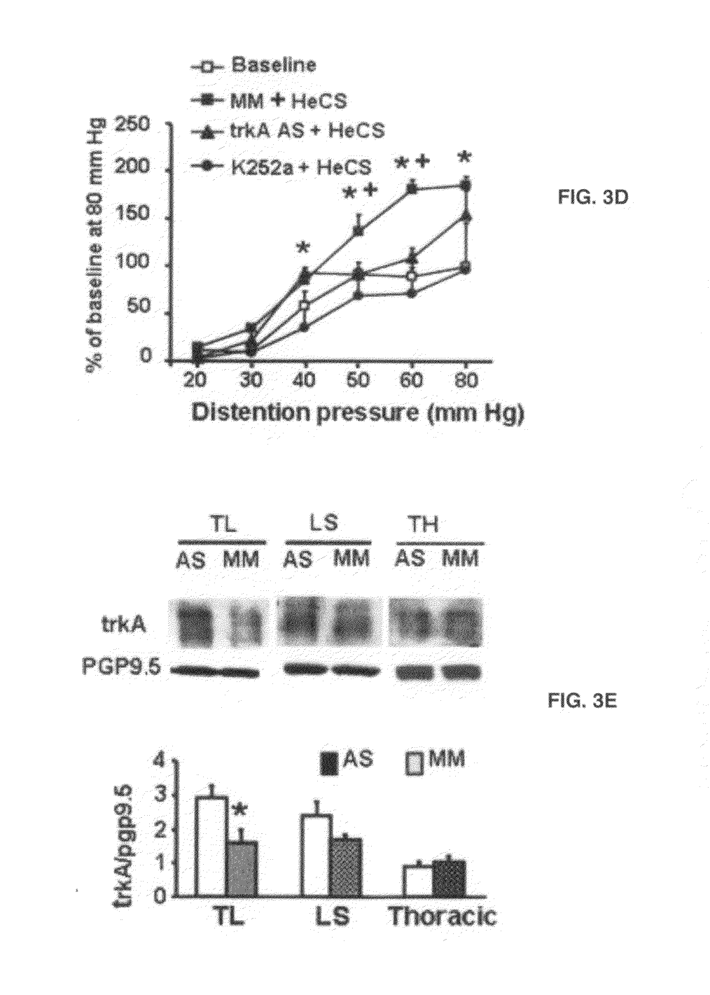

HeCS significantly increases the expression of nerve growth factorin the muscularis external serosa (ME/S) and in mucosa/submucosa (M/SM) of the distal colon, when compared with those in sham-stressed rats (FIG. 3A). Immunohistochemical staining of cross-sections detected nerve growth factorIR in smooth muscle and myenteric plexus in ME/S and in epithelial cells in M/SM. nerve growth factorIR increased in both tissue-types of the chronically stressed rats, compared with and sham-stressed rats (FIG. 3B). By contrast, heterotypic chronic stress had no significant effect on the expression of trkA receptors in ME/S (100±11% vs 108±27%) or M/SM (100±17% vs 92±5%).

### Example 13

### Effect of Antagonism of Peripheral Nerve Growth Factor on Stress-Induced Increase in Viscero-Motor Response

The rats treated with 16 μg/kg i.p. nerve growth factor antibody (14), 30 minutes before each daily stress session, showed no significant increase in viscero-motor response in response to colorectal distension following heterotypic chronic stress, compared to their pre-stress baseline responses (FIG. 3C). Rats receiving nonimmune serum showed significant increase in viscero-motor response following heterotypic chronic stress (FIG. 3C).

### Example 14

### Effects of Antagonism of trkA Receptors in TL Dorsal Root Ganglia on HeCS-Induced Increase in Excitability of TL Colon Afferent Neurons

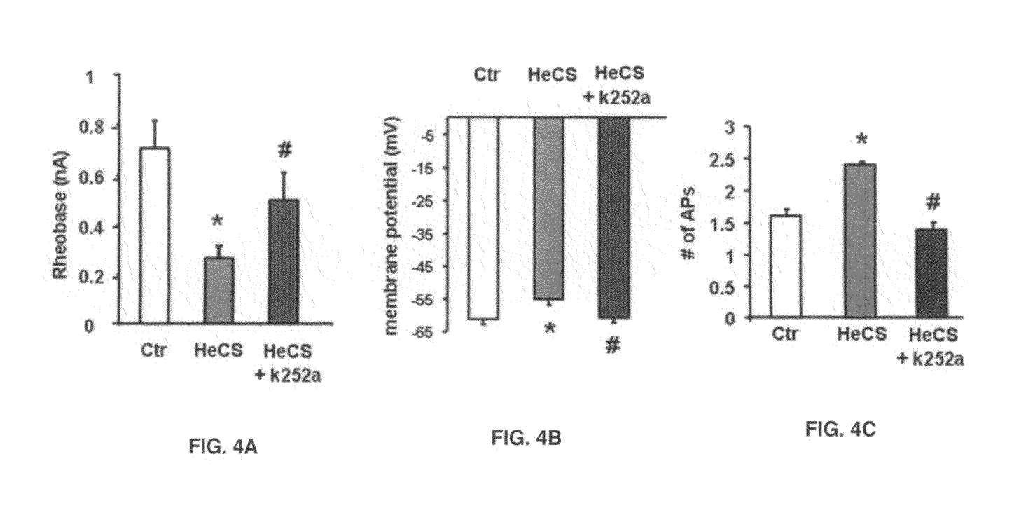

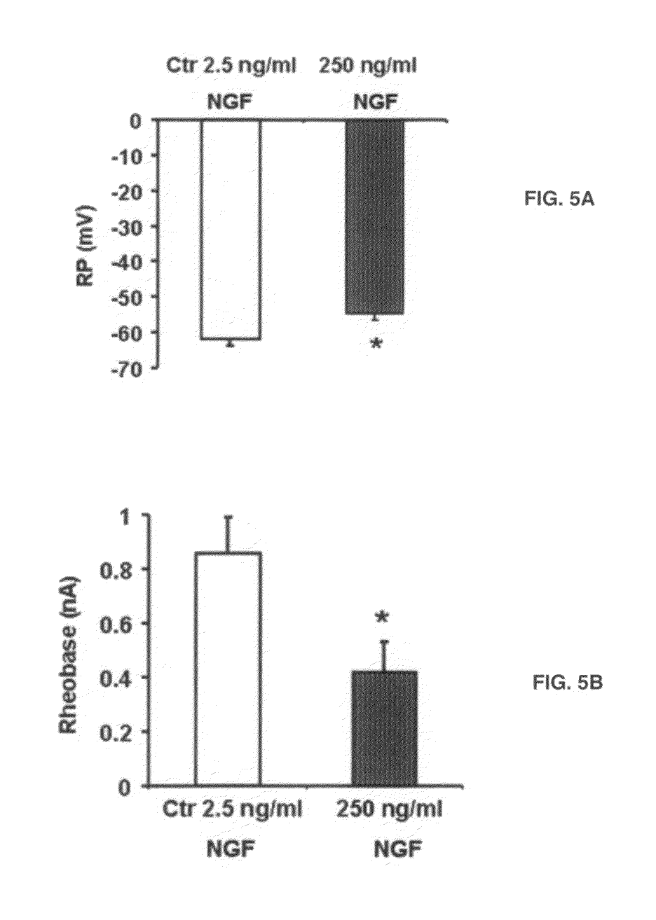

Next, whether nerve growth factor high affinity receptor trkA in dorsal root ganglia mediates the heterotypic chronic stress-induced increase in viscero-motor response to colorectal distension was examined. Either trkA pharmacological inhibitor k252a, or trkA anti-sense (AS) oligonucleotide (ODN) was administered intrathecally by a surgically implanted osmotic pump for the entire nine-day period of heterotypic chronic stress. The intrathecal infusions started 24-hours prior to the first session of heterotypic chronic stress. The control rats received infusion of vehicle or mismatch (MM) oligonucleotide. The rats treated with intrathecal k252a showed no significant difference between their post-HeCS and baseline responses to colorectal distension (FIG. 3D). The control rats that received MM oligonucleotide during stress showed significant increase in viscero-motor response to colorectal distension following HeCS compared to baseline responses (FIG. 3D). There was no significant difference between the post-HeCS and baseline responses of rats treated intrathecally with the trkA AS oligonucleotide. The expression of trkA in TL dorsal root ganglia of rats treated with anti-sense oligonucleotide decreased significantly to about 50% of that in rats treated with MM oligonucleotide (FIG. 3F). No significant difference in trkA levels were observed in thoracic dorsal root ganglion, several segments rostral to the catheter tip, or in LS DRG, several segments caudal to the catheter tip, indicating that the knockdown effect was localized to the dorsal root ganglia near the catheter tip (FIG. 3F). Intrathecal application of k252a significantly attenuated the heterotypic chronic stress-induced decrease in rheobase (FIG. 4A), depolarization of resting membrane potential (FIG. 4B), and increase in the number of action potentials at 2× rheobase (FIG. 4C). Taken together, these findings support an essential role of nerve growth factorreceptor trkA and nerve growth factorin colon-specific TL dorsal root ganglia neurons in the induction of visceral hypersensitivity by heterotypic chronic stress.

### Example 15

### The Role of Norepinephrine in Inducing Visceral Hypersensitivity and Nerve Growth Factor Expression in Distal Colon

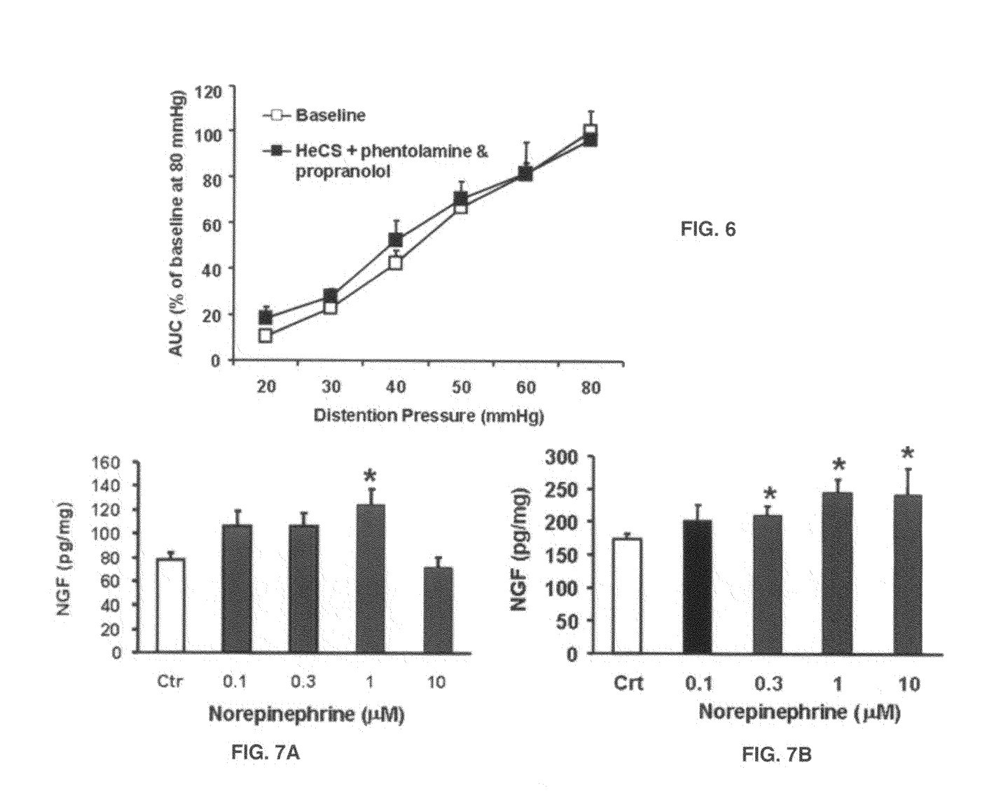

A nine-day HeCS significantly elevates plasma concentration of norepinephrine (4). To determine whether norepinephrine contributes to the induction of visceral hypersensitivity, rats subjected to HeCS were treated once daily before each stress session with phentolamine (2 mg/kg i.p.)+propranolol (2 mg/kg i.p.). Rats treated with phentolamine+propranolol exhibited no significant change in their viscero-motor response to colorectal distension compared to their respective pre-stress baselines (FIG. 6). Phentolamine plus propranolol also blocked the HeCS-induced increase in nerve growth factor in muscularis externa/serosa and mucosa/submucosa (FIG. 3A). Strips of muscularis externa or mucosa/submucosa were incubated with norepinephrine for 24 hours in vitro. Norepinephrine (FIGS. 7A-7B) concentration-dependently increased the expression of nerve growth factorin muscularis externa and in mucosal/submucosal tissues.

### Example 16

### The Roles of Inflammatory Mediators, Mast Cells and Peripheral CRH1/CRH2 Receptors in Induction of Visceral Hypersensitivity by HeCS

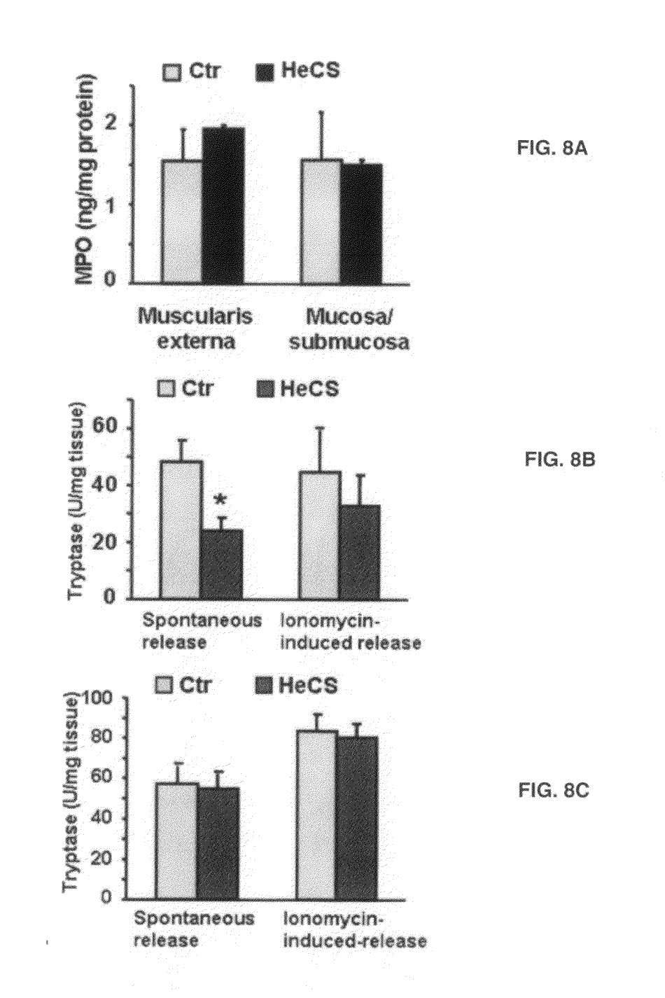

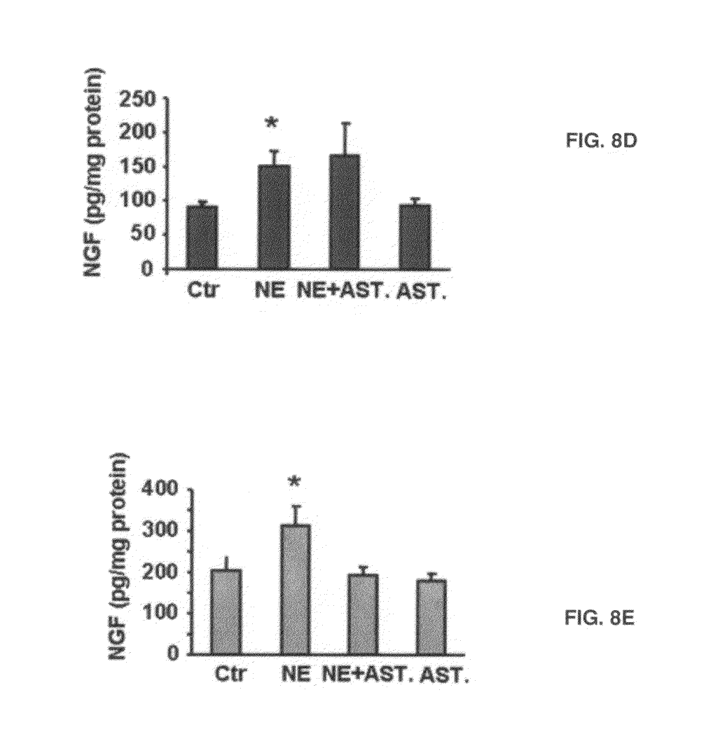

Nine-day heterotypic chronic stress had no significant effect on MPO in the muscularis externa or the mucosa/submucosa (FIG. 8A). It also did not alter the concentrations of TNFα and IL-1β in these tissues. Heterotypic chronic stress reduced the spontaneous release of tryptase from muscularis externa, but not that induced by ionomycin (FIG. 8B). Heterotypic chronic stress had no effect on spontaneous or ionomycin-induced release of tryptase in mucosal/submucosal tissues. Finally, the inhibition of CRH1/CRH2 receptors astressin had no significant effect on the generation of nerve growth factor by norepinephrine in muscularis externa (FIG. 8D), but astressin blocked this effect in the mucosa/submucosa (FIG. 8E).

### Discussion

The present invention shows that HeCS induces visceral hypersensitivity to colorectal distension that lasts for at least 8 hours, but it returns to baseline by 24 hours. Previously, it was found that heterotypic chronic stress induces colonic circular smooth muscle hyperreactivity to ACh that also lasts for about 8 hours and returns to baseline by 24 hours (4). Adrenergic stimulation mediates the induction of visceral hypersensitivity by heterotypic chronic stress. The blockade of α1/α2- and β1/β2-adrenergic receptors before the daily application of stress prevented the induction of visceral hypersensitivity by heterotypic chronic stress. By contrast, it was discovered that 10-day homotypic chronic stress (WAS) induces visceral hypersensitivity that lasts for about 40 days (9). However, they did not investigate which peripheral stress hormone/neurotransmitter mediates the prolonged induction of hypersensitivity in their model. Note that plasma/urine levels of norepinephrine are elevated in IBS patients (15-16).

The mechanoreceptors in the gut wall are distributed throughout its thickness (17-20). Accumulating evidence from human and animal studies shows that splanchnic neurons with mechanoreceptors in the muscularis externa/serosa mediate afferent responses to rapid step distensions that mimic the physiological rapid compression of the gut wall by giant migrating contractions (21) or migrating motor complexes (22). In vitro, the mucosal afferents respond primarily to stimulation by von frey hairs that mimic the flow of digesta (18-19); they do not respond to circumferential stretch (18). In vivo, the mucosal afferents respond to slow distension that mimics slow lumen filling by fecal material, but not to rapid step balloon distension 5. In addition, desensitization of the mucosal afferent mechanoreceptors by lidocaine does not affect pain-perception to rapid balloon distension in IBS patients (5). However, lidocaine blocks the sensory perception to slow distension. Therefore, the effects of heterotypic chronic stress on the expression of various mediators of DRG-sensitization were examined separately in the muscularis externa/serosa and mucosa/submucosa. Western blotting and immunohistochemical staining with nerve growth factorantibody showed that heterotypic chronic stress enhances the expression of nerve growth factorin ME/S as well as in M/SM. The inhibition of α1/α2-β1/β2 adrenergic receptors blocks increases in the expression of nerve growth factorin both tissues, suggesting that they mediate the expression of nerve growth factorin the colon wall in response to heterotypic chronic stress. In addition, in vitro incubation of both tissue-types with norepinephrine enhances the expression of NGF. Prior reports show that numerous cell-types, including smooth muscle cells (23), glia (24), immune cells (25) epithelial cells (26) and neurons (27) are capable of generating NGF.

In present invention, the smooth muscle cells and mucosa seemed to show the largest increase in nerve growth factor IR, but we did not quantitate it. Neutralization of peripheral nerve growth factor by its antibody blocked the increase of viscero-motor response to colorectal distension. Together, the above data suggests that the up regulation of nerve growth factor throughout the thickness of the distal colon wall by heterotypic chronic stress-induced release of norepinephrine is an intermediate step in the induction of visceral hypersensitivity to colorectal distension. Nerve growth factor in the periphery complexes with trkA receptors and migrates retrograde to the dorsal root ganglia neurons (28). The inhibition of retrograde migration by desensitization of afferent nerve endings by RTX blocked the induction of visceral hypersensitivity to colorectal distension. The pharmacological blockade of trkA receptors or their suppression by AS ODN in the TL dorsal root ganglia also blocked the induction of visceral hypersensitivity to colorectal distension. Taken together, nerve growth factor expression in the colon wall is critical for induction of visceral hypersensitivity by heterotypic chronic stress. Patch-clamp recordings from colon-specific TL dorsal root ganglia neurons show that heterotypic chronic stress decreases rheobase, depolarizes resting membrane potential and increases the electrogenesis of action potentials, when compared with those in age-matched shamstressed controls. Systemic administration of nerve growth factor antibody that does not cross the blood-brain barrier blocked these effects. This suggests that the alterations in the electrophysiological characteristics of colon-specific dorsal root ganglia neurons may primarily be due to increase of nerve growth factorin the colon wall, rather than due to a direct effect of plasma norepinephrine on the dorsal root ganglia neurons (29) or due to descending inhibition from the CNS (30).

Voltage-gated sodium channels (Nav) play a critical role in regulating the sensitivity of dorsal root ganglia neurons to peripheral insult. The C-type dorsal root ganglia neurons express predominantly the slowly inactivating TTX-R Nav1.8 and persistently active TTX-R Nav1.9 channels (31-32). The Nav1.8 channels make major contribution to the electrogenesis of action potentials (31. 33), whereas the Nav1.9 channels regulate the resting membrane potential of these neurons (32). The ionic mechanisms underlying visceral hypersensitivity are specific to the pathophysiology of pain and its targeted organ (35). Visceral hypersensitivity in rat colonic inflammation results mainly from alterations in the electrogenesis of action potentials due to remodeling of the Nav1.8 channels (36).

The present invention shows that visceral hypersensitivity induced by heterotypic chronic stress in rats is due to alterations in the electrogenesis of action potentials and changes in the resting membrane potentials of the colon-specific dorsal root ganglia neurons. According to the above established roles of Nav1.8 and Nav1.9 in regulating the electrophysiological characteristics of the dorsal root ganglia neurons, heterotypic chronic stress may remodel both types of Nav channels. The persistence of hypersensitivity in isolated dorsal root ganglia neurons for at least 8-hours after the last stressor indicates altered gene expression of the Nav channels (37-38). Nerve growth factor is a key signaling protein in the sensitization of visceral afferent neurons during inflammation (39-42). The administration of exogenous nerve growth factor mimics inflammatory hyperalgesia 42, while systemic administration of nerve growth factorantibody blocks the induction of inflammatory hyperalgesia (14). During inflammation, nerve growth factor interacts with mast cells in a reciprocal synergistic reaction (11, 25). Nerve growth factor acts on trkA receptors on mast cells to degranulate them, while the degranulation of mast cells releases NGF. It is noteworthy that while both nerve growth factor and trkA receptors are up regulated during inflammation (42), only nerve growth factor expression is enhanced in the muscularis externa by non-inflammatory stress mediator norepinephrine.

Other studies found increase in mast cell numbers or their activation and increase in MPO activity and proinflammatory cytokines in mucosa/submucosa in response to chronic stress (8-9) or maternal deprivation (43). The consensus seems to be that the overall intensity of inflammatory responses in these models is several-fold less than that in models of TNBS-induced inflammation. By contrast, there was no evidence of an increase in MPO, proinflammatory cytokines or mast cell activation/hyperplasia in the muscularis externa or mucosa/submucosa in response to heterotypic chronic stress, which induces robust visceral hypersensitivity to colorectal distension. In addition, nerve growth factor increases in both tissue-types in response to heterotypic chronic stress, which was blocked by inhibiting α1/α2-β1/β2-adrenergic receptors. Norepinephrine increases the expression of nerve growth factor in vitro in muscularis externa mucosa/submucosa, and this increase is blocked by inhibition of adrenergic receptors. Taken together, norepinephrine induces the expression of nerve growth factorin the colon wall in the absence of any detectable inflammatory response.

CRH receptors have been identified in the mucosa as well as in the myentreric and submucosal plexi (44-45). These findings show that these receptors in the muscularis externa that includes the myenteric and submucosal plexi do not mediate the generation of nerve growth factor by norepinephrine. However, the CRH receptors in the mucosa mediate the expression of nerve growth factorin mucosal/submucosal tissues. In conclusion, 9-day heterotypic chronic stress induces visceral hypersensitivity to colorectal distension that lasts for at least 8-hours after the last stressor. Heterotypic chronic stress does not induce an inflammatory response in the muscularis externa or the mucosa/submucosa. It was reported previously that heterotypic chronic stress elevates the plasma level of norepinephrine that alters gene expression of key cell signaling proteins of the excitation-contraction coupling in colonic circular smooth muscle cells to induce hyperreactivity to acetylcholine (4). The present invention shows that the increase in plasma concentration of norepinephrine also mediates the induction of nerve growth factorin muscularis externa and mucosa/submucosa. Based on the topology and phenotypes of afferent nerve endings in the colon wall (5, 17-20), increase of nerve growth factorin muscularis externa may mediate the induction of visceral hypersensitivity by heterotypic chronic stress, whereas, increase of nerve growth factor in mucosa/submucosa may mediate altered physiological responses to digesta in the lumen. The retrograde transport of NGF-trkA complex sensitizes TL dorsal root ganglia decreasing rheobase, depolarizing resting membrane potential, and increasing the electrogenesis of action potentials.

The following references are cited herein:
1. Bennett et al., Gut 1998; 43:256-61.
2. Whitehead et al., Gut 1992; 33:825-30.
3. Choudhury et al., Am J Physiol Gastrointest Liver Physiol 2009; 296:G632-42.
4. Choudhury et al., Am J Physiol Gastrointest Liver Physiol 2009.
5. Lembo et al., Gastroenterology 1994; 107:1686-96.
6. Lembo et al., Dig Dis Sci 1997; 42:1112-20.
7. Carrasco et al., Eur J Pharmacol 2003; 463:235-72.
8. Gue et al., Neurogastroenterol Motil 1997; 9:271-9.
9. Bradesi et al., Am J Physiol Gastrointest Liver Physiol 2005; 289:G42-53.
10. Xu et al., Gut 2008; 57:1230-7.
11. Skaper et al., Brain Res Mol Brain Res 2001; 97:177-85.
12. Summer et al., J Pain 2006; 7:884-91.
13. Karai et al., J Clin Invest 2004; 113:1344-52.
14. Woolf et al., Neuroscience 1994; 62:327-31.
15. Heitkemper et al., Am J Gastroenterol 1996; 91:906-13.
16. Posserud et al., Gut 2004; 53:1102-8.
17. Leek BF. Abdominal and pelvic visceral receptors. Br Med Bull 1977; 33:163-8. 18.
18. Lynn et al., J Physiol 1999; 518 (Pt 1):271-82.
19. Brierley et al., Gastroenterology 2004; 127:166-78.
20. Sengupta et al., J Neurophysiol 1994; 71:2046-60.
21. Sarna SK. Am J Physiol Gastrointest Liver Physiol 2007; 292:G572-81.
22. Kellow et al., Gastroenterology 1991; 101:1621-7.
23. Clemow et al., J Cell Physiol 2000; 183:289-300.
24. von Boyen et al., J Neuroendocrinol 2006; 18:820-5.
25. Leon et al., Proc Natl Acad Sci USA 1994; 91:3739-43.
26. Stanzel et al., Exp Neurol 2008; 211:203-13.
27. Kobayashi et al., J Pediatr Surg 1999; 34:543-8.
28. Barker et al., Trends Neurosci 2002; 25:379-81.
29. Khasar et al., J Neurophysiol 1999; 81:1104-12.
30. Pyner et al., Neuroscience 1998; 83:617-31.
31. Sangameswaran et al., J Biol Chem 1996; 271:5953-6.
32. Dib-Hajj et al., Trends Neurosci 2002; 25:253-9.
33. Renganathan et al., J Neurophysiol 2001; 86:629-40.
34. Herzog et al., J Neurophysiol 2001; 86:1351-64.
35. Gold M S, et al., J Neurophysiol 2004; 91:2524-31.
36. Beyak et al., Am J Physiol Gastrointest Liver Physiol 2004; 287:G845-55.
37. Gould et al., Brain Res 2000; 854:19-29.
38. Fjell et al., J Neurosci Res 1999; 57:39-47.
39. Aloe et al., Rheumatol Int 1992; 12:213-6.
40. Chien et al., J Pain 2007; 8:161-7.
41. Winston et al., J Pain 2003; 4:329-37.
42. Delafoy et al., Pain 2003; 105:489-97.
43. Barreau et al., Gastroenterology 2004; 127:524-34.
44. Chatzaki et al., J Neurochem 2004; 90:309-16.
45. Liu et al., J Comp Neurol 2006; 494:63-74.

What is claimed is:

1. A method of treating an individual with irritable bowel syndrome, comprising:
reducing hypersensitivity of dorsal root ganglia neurons in the individual.

2. The method of claim 1, wherein said reduction of hypersensitivity is achieved by administering resiniferatoxin to the individual.

3. The method of claim 2, wherein said administering is luminal administration of resiniferatoxin in distal colon.

4. The method of claim 3, wherein said administration is intraluminal infusion of about 200 ug/kg of resiniferatoxin.

5. A method of treating an individual with irritable bowel syndrome, comprising the step of:
inhibiting nerve growth factor in the individual.

6. The method of claim 5, wherein said inhibition is achieved by administering an nerve growth factor antibody to the individual.

7. The method of claim 5, wherein said inhibition is achieved by administering an inhibitor of trkA receptors in the individual.

8. The method of claim 7, wherein said inhibitor is k252A, astressin or trkA anti-sense oligonucleotide.

9. The method of claim 8, wherein said anti-sense oligonucleotide has the sequence shown in SEQ ID NO: 1.

10. The method of claim 5, wherein said inhibition is achieved by blocking β1/β2- and β1/β2/β3-adrenergic receptors in the individual.

11. The method of claim 8, wherein said inhibitor is administered intrathecally.

12. A method of treating an individual with irritable bowel syndrome, comprising:
administering inhibitor of norepinephrine to the individual.

13. The method of claim 12, wherein said inhibitor is phentolamine, propranolol, cyanopindolol or a combination thereof.

14. The method of claim 12, wherein said inhibitor is injected intraperitoneally.

15. The method of claim 13, wherein the dosage of said inhibitor is about 2 mg/kg.

16. A method of treating an individual with irritable bowel syndrome, comprising:
reducing hypersensitivity of dorsal root ganglia neurons in the individual;
inhibiting nerve growth factor in the individual;
administering inhibitor of norepinephrine to the individual; or
a combination thereof.

17. The method of claim 16, wherein said reduction of hypersensitivity is achieved by administering resiniferatoxin to the individual.

18. The method of claim 17, wherein said administering is luminal administration of resiniferatoxin in distal colon.

19. The method of claim 18, wherein said administration is intraluminal infusion of about 200 μg/kg of resiniferatoxin.

20. The method of claim 16, wherein said inhibition of nerve growth factor is achieved by administering a nerve growth factor antibody to the individual.

21. The method of claim 16, wherein said inhibition of nerve growth factor is achieved by administering an inhibitor of trkA receptors in the individual.

22. The method of claim 21, wherein said inhibitor is k252A, astressin or trkA anti-sense oligonucleotide.

23. The method of claim 22, wherein said anti-sense oligonucleotide has the sequence SEQ ID NO: 1.

24. The method of claim 16, wherein said inhibition of nerve growth factor is achieved by blocking α1/α2- and β1/β2-adrenergic receptors in the individual.

25. The method of claim 22, wherein said inhibitor is administered intrathecally.

26. The method of claim 24, wherein said inhibitor of noepinephrine is phentolamine, propranolol or a combination thereof.

27. The method of claim 26, wherein said inhibitor is injected intraperitoneally.

28. The method of claim 26, wherein the dosage of said inhibitor is about 2 mg/kg.
